(19) United States
(12) Patent Application Publication
(10) Pub. No.: US 2019/0022342 A1
(43) Pub. Date: Jan. 24, 2019

(54) Method and Device for Ventilating a Patient

(71) Applicant: Ventinova Technologies B.V., Eindhoven (NL)

(72) Inventor: Dietmar Enk, Coesfeld (DE)

(21) Appl. No.: 16/081,600

(22) PCT Filed: Jan. 31, 2017
(86) PCT No.: PCT/EP2017/052001

§ 371 Date: Aug. 31, 2018

(30) Foreign Application Priority Data
Mar. 1, 2016 (DE) 10 2016 103 678.1
May 24, 2016 (DE) 10 2016 109 528.1

(51) Int. Cl. A61M 16/00

(52) U.S. Cl. CPC A61M 16/024; A61M 16/04; A61M 16/0003; A61M 16/0051

(57) ABSTRACT

The invention relates to a ventilation device for ventilating a patient, comprising at least a fluid supply unit or, additionally, a fluid discharge unit which can be used to guide a fluid into at least one airway, that is part of a lung or in a lung, of a patient or for discharging the fluid from said airway; and to a control device which, during the ventilation of at least one airway of the patient, can be used to guide a fluid into the at least one airway and/or discharge the fluid from at least one airway by operating the ventilation device, for determining or also for estimating a path of at least one region of a compliance curve of the at least airway by guiding and/or discharging the fluid from the at least one airway and by determining at least one value of the compliance. The invention also relates to a method for operating a ventilation device.

The subject matter of the present invention relates to a ventilation device and to a method for ventilating a patient. The device comprises at least a fluid supply unit or, additionally, a fluid discharge unit which can be used to supply a fluid (respiratory gas) into at least one airway, that is to say into a lung part or into the lung, of a patient or for discharging the fluid from this airway.

When a patient is being ventilated, a mask or a tube is normally used via which a gas or gas mixture, in particular oxygen and air, is supplied at low pressure to the airway sealed off from the outside. Alternatively, however, a gas or gas mixture of this kind can also be injected in pulses at a high pressure and a high flow rate through a thin, unblocked catheter into the airway lying open to the outside (jet ventilation). This method is nowadays used particularly in diagnostic and therapeutic interventions in the region of the upper airway (endotracheal or transtracheal jet ventilation). This method can also be applied in emergency situations outside the hospital environment or in inpatient situations within hospitals.

In transtracheal jet ventilation, a patient can be supplied with oxygen or a fluid by way of a catheter that has been introduced directly into the trachea through the skin or by way of a cannula thus placed. These methods (transtracheal/endotracheal) are constituent parts of the currently valid algorithms for managing difficult airways and, in particular, the situation in which a patient cannot be ventilated or cannot be intubated by conventional means (what is called a “cannot ventilate, cannot intubate” situation).

Moreover, WO 2008/113752 A1 and WO 2015/004229 A1 each disclose gas flow reversing devices with which ventilation (inhalation and exhalation) can also take place exclusively via a catheter.

Artificial or mechanical ventilation takes place either in a controlled manner or in the form of assisted spontaneous ventilation. In the former case, the respirator has complete control over the breathing pattern, whereas in the latter case the at least partially spontaneously breathing patient has considerable influence over the breathing pattern. However, a common aspect of all forms of ventilation is that the respirator almost exclusively influences the inhalation phase. From the perspective of the respirator, the exhalation can take place passively, i.e. the energy stored in the elastic tissue elements of lung and thorax drives the exhalation.

Various methods of ventilation are known. Usually, volume-controlled ventilation is performed, in which all the ventilation parameters are predefined. The target parameter and control parameter is the tidal volume V_{T}. The resulting airway pressures are dependent on the volumes that are set and on the conditions of the patient's pulmonary system. Adjustment parameters are therefore volumetric flow, ventilation frequency and PEEP (positive end-expiratory pressure). The positive end-expiratory pressure denotes a positive pressure which is generated artificially in the lungs during ventilation and which is present after completion of the exhalation.

In pressure-controlled ventilation, an initially high volumetric flow is reduced only after a high inhalation pressure level that has been set is reached. The target parameter and control variable is therefore the pressure. An adjustment of the volumetric flow is therefore not possible here.

In contrast to the spontaneous ventilation of a patient, the fluid in artificial ventilation is supplied counter to the elasticity of the airway. As a result of the increased pressure in the thorax, PEEP reduces the return flow of the venous blood to the heart, as a result of which the cardiac output can drop. Conversely, congestion occurs in the superior and inferior vena cava, with corresponding pressure increases in the upstream organs. Depending on the level of the PEEP, this can lead to damage and functional impairment of the brain, liver, kidneys and other organs.

Proceeding from this, the object of the present invention is to propose an improved ventilation method. In particular, the ventilation is intended to take place in a manner that is tailored to the individual as far as possible, i.e. the characteristics of the patient to be ventilated are to be taken fully into consideration. Moreover, the ventilation should be as gentle as possible, and damage to the airways and other organs must be prevented in every case. Moreover, a ventilation device is to be proposed which allows such ventilation to be carried out.

This object is achieved by ventilation devices having the features of claims 1 and 9 and also by methods having the features of claims 13 and 22. Advantageous developments and embodiments of the ventilation devices and of the methods are the subject matter of the respective dependent claims. It will be noted that the features specified individually in the dependent claims can be combined with one another in any desired technologically meaningful way and define further embodiments of the invention. Furthermore, the features specified in the claims are rendered more precisely and explained in more detail in the description, with further preferred embodiments of the invention being presented.

The invention relates to a (first) ventilation device for ventilating a patient, at least comprising a fluid supply unit or, additionally, a fluid discharge unit, which is suitable for supplying a fluid into at least one airway, that is to say into a lung part or into the lung, of a patient or for discharging the fluid from this airway. Moreover, the ventilation device comprises a control device which, during a ventilation of the at least one airway of the patient, that is to say during the supply of a fluid into the at least one airway and/or the discharge of the fluid from the at least one airway by operation of the ventilation device, is suitable for determining or in addition estimating a profile of at least one subregion of a compliance curve of the at least one airway. The determination or the additional estimation of a profile of at least one subregion of a compliance curve takes place by supplying and/or discharging the fluid from the at least one airway and by determining at least one value of the compliance.

The following applies for the compliance:

C=V/delta p [milliliter/millibar].

The compliance indicates how much fluid, that is to say a volume V [milliliter], is introduced into the at least one airway or is removed from the airway, such that a pressure in the airway changes by a pressure difference delta p [millibar]. The control device, taking into account the determined or additionally estimated profile of the at least one subregion of the compliance curve, determines a position of a pressure interval with the pressures P1 and P2 and sets these pressures on the ventilation device such that at least one ventilation process, that is to say an inhalation and/or an exhalation, takes place between these pressures P1 and P2 and an absolute value of the compliance of this ventilation process is as high as possible.

The absolute value indicates the value of the compliance independently of its sign.

The underlying concept of the invention is to perform ventilation of the patient with the lowest possible energy input. A low energy input into the airways of the patient also signifies the least possible damage to the airways and other organs of the patient. This minimizing of the energy input is achieved through the determination of the lowest possible pressure at which a required tidal volume V_{T} can be supplied to the patient. These pressures P1 and P2 of the pressure interval are determined on the basis of the respectively existing compliance of the ventilated patient.

In particular, the profile of the compliance curve is thus determined or additionally estimated (e.g. on the basis of empirical values) during at least one ventilation process (inhalation and/or exhalation). In particular, it is specifically the subregion of the compliance curve at which a defined volume V (or V_{T}) can be supplied in the smallest possible pressure interval that is determined.

In particular, in order to determine the profile of the compliance curve, a volume of fluid, preferably a small volume of at most 100 ml, particularly preferably of at most 50 ml, is supplied to the at least one airway via the fluid supply unit. During and/or preferably after the supply of this volume, the pressure change delta p in the at least one airway is measured and a value for the compliance is determined. At least the profile of the subregion of the compliance curve is then estimated, either taking into consideration empirical values or, if appropriate, taking into consideration values that have already been determined for the compliance of this patient. Alternatively, further (small) volumes are supplied and the respective pressure change delta p determined. From these values for the compliance, the profile of at least the subregion of the compliance curve can be determined and/or estimated (with increasing precision). Moreover, the profile of the compliance curve and the preferred position of a pressure interval, provided for the subsequent ventilation of the patient, with the pressures P1 and P2 can be determined or estimated on the basis of decreasing or increasing absolute values of the compliance values.

In particular, at least one of the following variables—PEEP, respiration rate, volumetric flow—can be preset, such that the required tidal volume V_{T} can be supplied, on condition of the lowest possible energy input. Preferably, each of these variables can also be further adapted, after determination and evaluation of the ventilation process, such that a predetermined tidal volume V_{T} is supplied under the parameters that are then set.

A fluid supply unit, and optionally also a fluid discharge unit, comprises at least one source of compressed gas or a device with which a fluid (e.g. a gas or a gas mixture suitable for ensuring the ventilation of a patient) can be introduced into and removed from the at least one airway of the patient. Preferably, a source of compressed gas is exclusively present, or the exhalation also takes place via a gas flow reversing device as mentioned above, wherein the fluid is introduced into the airway via a lumen and is discharged again via the same lumen.

The control device is suitable for determining or additionally for estimating a profile of at least one subregion of a compliance curve. The determination of the compliance curve takes place during a ventilation by the supply and/or discharge of the fluid from the at least one airway and by determination of at least one value of the compliance. The profile of the compliance curve of a patient can in particular be estimated taking into consideration the at least one value of the compliance.

In particular, the control device falls back on the measured values of at least one pressure sensor and monitors the volumetric flows that are supplied via the fluid supply unit and that are discharged via the fluid discharge unit.

In particular, the pressure present in the respective airway is monitored and/or measured and computationally estimated or determined. A pressure sensor is thus preferably arranged in the airway, such that a continuous pressure measurement can in particular also take place in the airway even during the ventilation.

Such an arrangement of a pressure sensor is particularly advantageous in the determination of the profile of the compliance curve, since in this case the (respectively) changing pressure delta p can be determined during the continuous or staged supply of a volume or of partial volumes of the fluid.

According to a preferred embodiment, the control device, at least during an inhalation or an exhalation of a ventilation process, determines a plurality of values for the compliance and from these determines, for at least one subsequent ventilation process, the position of the pressure interval with the pressures P1 and P2, for which an absolute value of the compliance is as high as possible. In particular, the control device determines the values for the compliance continuously or at predefinable time intervals. At least 5 values of the compliance, particularly preferably at least 10 values of the compliance, are preferably determined for each ventilation process.

In particular, the number of compliance values to be determined for each ventilation process is adjustable. Preferably, a different number of values is determined for an inhalation than is determined for an exhalation.

In particular, values for the compliance are determined by the control device across a plurality of ventilation processes continuously (that is to say not only during one ventilation process but during each ventilation process), such that the position of the pressure interval with the pressures P1 and P2 can optionally be determined anew for each subsequent ventilation process or for a plurality of successive ventilation processes.

In particular, the control unit, as a function of the determined position of the pressure interval, of the pressure interval itself and of the determined compliance, determines at least for the subsequent ventilation process at least one of the following parameters:
a tidal volume V_{T} [milliliter],
a pressure P1 and a pressure P2 [millibar],
a ventilation frequency F [1/second].

According to a preferred embodiment of the ventilation device, at least one pressure sensor can be arranged inside the airways of the patient, and the pressure in the airway can be determined by a measurement inside the at least one airway.

At least the pressure rise, that is to say delta p/delta t [millibar/second], during an inhalation can preferably be controlled and limited by the ventilation device.

According to a preferred embodiment of the ventilation device, at least the pressure drop, that is to say delta p/delta t [millibar/second], during an exhalation can be controlled and limited by the ventilation device.

According to a preferred embodiment of the ventilation device, the pressure rise and the pressure drop can be controlled and limited.

Preferably, the absolute value of the pressure rise or pressure drop can be limited to at most 40 mbar/s [millibar/second], in particular at most 30 mbar/s, preferably at most 20 mbar/s.

Moreover, a (first) method is proposed for operating a ventilation device, in particular the ventilation device according to the invention. The ventilation device is provided for ventilating a patient. The method comprises at least the following steps:
a) supplying a fluid into at least one airway, that is to say a lung part or the lung, of the patient and/or discharging the fluid from this airway by operation of the ventilation device;
b) determining or additionally estimating a profile of at least one subregion of a compliance curve of the at least one airway by the supply and/or discharge of the fluid in step a) and determining at least one value of the compliance, wherein the following applies for the compliance:

C=V/delta p [milliliter/millibar];
wherein the compliance indicates how much fluid, that is to say a volume V [milliliter], is introduced into the at least one airway or is removed from the airway, such that a pressure in the airway changes by a pressure difference delta p [millibar];
c) determining a position of a pressure interval with the pressures P1 and P2 along the profile of the at least one subregion of the compliance curve determined or additionally estimated in step b), wherein an absolute value of the compliance is as high as possible for a ventilation process performed in this pressure interval, that is to say an inhalation and/or an exhalation;
d) supplying and/or discharging the fluid within the pressure interval determined in step c), in at least one ventilation process following on from step c).

The statements concerning the ventilation device apply equally to the method proposed here, and vice versa.

A method is thus proposed for ventilating a patient with the lowest possible energy input. The minimizing of the energy input is achieved through the determination of the lowest possible pressure at which a required tidal volume V_{T} can be supplied to the patient. These pressures P1 and P2 of the pressure interval are determined on the basis of the respectively existing compliance of the ventilated patient.

In particular, in step b), a plurality of values for the compliance are determined at least during an inhalation or an exhalation of a ventilation process, such that in step c) it is possible to determine, for at least one subsequent ventilation process, the position of the pressure interval with the pressures P1 and P2 for which an absolute value of the compliance is as high as possible. In particular, the control device determines the values for the compliance continuously or at predefinable time intervals. At least 5 values of the compliance, particularly preferably at least 10 values of the compliance, are preferably determined for each ventilation process.

Steps b), c) and d) are preferably carried out continuously, such that the position of the pressure interval with the pressures P1 and P2 is optionally newly determined for each subsequent ventilation process or for a plurality of successive ventilation processes.

According to a preferred embodiment, as a function of the position of the pressure interval determined in step c), of the pressure interval itself and of the determined compliance, at least one of the following parameters is determined at least for the subsequent ventilation process:
a tidal volume V_{T} [milliliter],
a pressure P1 and a pressure P2 [millibar],
a ventilation frequency F [1/second].

According to a preferred embodiment, the pressure is determined by a measurement inside the airways.

According to an advantageous embodiment, at least the pressure rise, that is to say delta p/delta t [millibar/second], during an inhalation is controlled and limited.

According to another advantageous embodiment, at least the pressure drop, that is to say delta p/delta t [millibar/second], during an exhalation is controlled and limited.

The pressure rise and the pressure drop are preferably controlled and limited.

In particular, the absolute value of the pressure rise or pressure drop is limited to at most 40 mbar/s [millibar/second], in particular at most 30 mbar/s, preferably at most 20 mbar/s.

In particular, the patient is ventilated using a catheter which has a cross section of at most 30 mm² [square millimeters] for the passage of at least one fluid supplied during the inhalation.

In particular, with such a small cross section (with inhalation and exhalation exclusively via this lumen), the pressure rise can be limited during the inhalation but also during the exhalation.

In particular, a resistance (e.g. a flow resistance or the like) can be provided in a fluid discharge unit and limits and controls the pressure drop during the exhalation.

For ventilation device and method, it applies equally that a subregion of a compliance curve present for the at least one airway of the patient to be ventilated is in particular initially determined and, if appropriate, additionally estimated. For this, the pressure rise during the delivery of a defined volume V (e.g. 50 or 100 ml [milliliter]; optionally also V_{T}) is measured.

Moreover, a PEEP level is in particular determined thereafter (that is to say the lower pressure of P1 and P2 ). To determine the PEEP level with which the patient is subsequently to be ventilated, several ventilation processes can initially also be carried out with in each case different PEEP levels.

Moreover, a tidal volume V_{T} anticipated for the patient in question is set. This tidal volume V_{T} can be further adapted during the ventilation, e.g. on the basis of monitoring the CO₂ level. Alternatively or additionally, the CO₂ level can also be influenced by means of the frequency of the ventilation processes.

In particular, the pressure rise and/or the pressure drop is controlled and monitored during the ventilation, such that the shear stress acting on the at least one airway is minimized.

In any case, however, the ventilation device and/or the method ensures that an absolute value of the compliance during a ventilation process is as high as possible, or in particular in other words that
(1) the ventilation takes place in a pressure interval in which the supplied volume of the fluid is maximal, or
(2) the supply or discharge of a predetermined volume V or of a tidal volume V_{T} of the fluid takes place within a pressure interval that is as small as possible.

The invention relates to a further (second) method for ventilating a patient and/or for operating a ventilation device, in particular the ventilation device according to the invention. The ventilation device is provided for ventilating a patient.

The second method is also directed to the ventilation of a patient, wherein the lowest possible energy input into the airways of the patient is to be achieved. According to the second method, the fluid supplied during the inhalation of the patient's lung or discharged during the exhalation from the patient's lung by the ventilation device is controlled actively and continuously (that is to say at each point in time) during the ventilation of the patient. The active control comprises a continuous pressure change of the supplied and discharged fluid by the ventilation device. The continuously changed pressure is in particular the pressure inside the airways and thus inside the lung. This pressure can be determined by a sensor via a measurement at the end of a ventilation device, e.g. a catheter, that reaches into the airway.

The continuous pressure change leads in particular to a continuous control of the fluid supply rate and fluid discharge rate [milliliter/second] through the ventilation device to the lung or from the lung during the ventilation processes. In particular, the fluid volume present in the lung is thus continuously changed. During the change of the fluid volume located in the lung, the fluid supply rate and/or the fluid discharge rate through the ventilation device to the lung or from the lung are preferably not changed and thus remain substantially constant. The fluid supply rate does not necessarily have to correspond to the fluid discharge rate, although it can also be of the same magnitude. Moreover, the fluid supply rate can be varied from one inhalation process to the following inhalation process. The same applies, in particular independently thereof, to the fluid discharge rate during successive exhalation processes.

In particular, states are avoided in which there is no change of the pressure and in particular no change of the fluid volume present in the lung within a time interval. Preferably, such time intervals in which there is no change of the pressure and/or in particular no change of the fluid volume present in the lung are at most 0.5 s [seconds], in particular at most 0.2 s, preferably at most 0.1 s in length and in particular concern (exclusively) the time of reversal of the flow of fluid (that is to say the transition from fluid supply to fluid discharge, and vice versa).

The pressure is in particular measured in the patient himself, particularly advantageously in the region of the outflow from the ventilation device, that is to say from a lumen (tube/catheter) transporting the fluid, into the airway of the patient. Alternatively and/or additionally, the pressure is measured in the ventilation device.

In particular, the pressure in the ventilation device does not correspond to the pressure in the airway of the patient. In particular, a continuous change of the pressure in the airways can also be set by an at least intermittently constant pressure in the ventilation device.

A change of the pressure in the airways can in particular also still be measured when a fluid supply rate or fluid discharge rate is zero. This change results in particular from the properties of the airways themselves. However, the (second) method is geared toward the connection between fluid supply rate and fluid discharge rate (not equal to zero) and pressure change. A fluid supply rate and fluid discharge rate of zero should as far as possible be avoided (at most for time intervals of up to 0.5 s [seconds], in particular at most 0.2 s or 0.1 s, and then also only at the time of reversal of the flow of fluid; if appropriate, longer time intervals of up to 2.0 s are possible, e.g. in order to carry out a pressure measurement, wherein such an extended time interval is provided only at distances of at least 30 s, in particular at least 2 minutes, preferably at least 5 minutes). For this purpose, the fluid supply rate and fluid discharge rate is in particular predefined (exclusively) by the ventilation device, wherein the pressure in the airways is monitored.

In particular, a sinusoidal or sawtooth-shaped breathing pattern (pressure [millibar] over time [second]) is thus set, wherein a rise of the curve (pressure over time) is constantly not equal to zero, and it has a rise equal to zero in particular only at the time of reversal of the flow of fluid for a time interval of at most 0.5 s [seconds], in particular at most 0.2 s, preferably at most 0.1 s, particularly preferably never.

In the context of the second method, a breathing pattern is predefined for the patient preferably at all times during the ventilation by the ventilation device, i.e. the fluid supply rate (inhalation flow) and fluid discharge rate (exhalation flow) are controlled and determined (alone) by the ventilation device (and not by the patient).

In particular, the fluid supply and, if appropriate, additionally the fluid discharge take place exclusively via the ventilation device or via at least one lumen inserted into the airways of the patient.

The continuous pressure change ensures that the fluid supply and fluid discharge do not take place too quickly or too slowly, and it is thus possible to prevent or at least minimize damage to the airways and in particular to the lung tissue.

Moreover, the fluid supply and fluid discharge can take place, e.g. taking into consideration a compliance of the airways (see statements concerning the first method and the ventilation device, which can be transferred equally to the second method) at advantageous pressure intervals (that is to say between a first, higher pressure and a second, lower pressure) and at a predefinable ventilation frequency.

In particular, in the second method, a ventilation pause (i.e. no flow of fluid to or from the airway) of more than 0.5 s [seconds], in particular of more than 0.2 s, preferably of more than 0.1 s, is avoided, particularly preferably never. In the known ventilation methods, such ventilation pauses are provided in order to maintain a predefined ventilation rhythm (frequency and/or ratio of inhalation and exhalation) or in order to limit the fluid volume to be supplied (at a predefined pressure). Moreover, in the known ventilation methods, ventilation pauses also occur (randomly) on account of the characteristics of the lung (e.g. compliance) or are influenced by these. However, ventilation pauses have the consequence that a fluid supply or fluid discharge has to be intensified or carried out more quickly at other times (with a greater flow of fluid and higher energy input into the airways or the lung of the patient).

This problem is solved by the second method in that ventilation pauses are (largely) avoided and the fluid supply or fluid discharge can then be carried out at other times with a reduced flow of fluid and thus lower energy input into the airways or the lung of the patient.

Besides the (second) method, a (second) ventilation device is also claimed, in particular the (first) ventilation device according to the invention. The ventilation device is used to ventilate a patient and comprises at least a fluid supply unit or, additionally, a fluid discharge unit which is suitable for supplying a fluid into at least one airway, that is to say into a lung part or into the lung, of a patient or for discharging the fluid from this airway. The ventilation device moreover comprises a control device which, during ventilation of the at least one airway of the patient, that is to say during the supply of a fluid into the at least one airway and/or the discharge of the fluid from the at least one airway, is suitable for regulating the ventilation, that is to say in particular for controlling a profile of at least one of the following variables: pressure in the airway of the patient (e.g. by measurement in the ventilation device and if appropriate by assessment of the pressure in the airway; or by measurement in the airway of the patient), supply rate of the fluid, discharge rate of the fluid, volume in the airway, etc.

The control device regulates at least one ventilation process such that a pressure in the at least one airway is continuously changed at least during an entire inhalation or at least during an entire exhalation, by continuous control of a fluid supply rate or of a fluid discharge rate during the ventilation process.

In particular, the control device regulates at least one ventilation process such that there is a substantially constant fluid supply rate [milliliter/second] at least during an entire inhalation or there is a substantially constant fluid discharge rate [milliliter/second] at least during an entire exhalation.

Preferably, the control device regulates at least one ventilation process, which comprises at least one inhalation and one exhalation, such that a pressure in the at least one airway is changed continuously during the ventilation process.

In particular, a continuous change of the pressure entails that the pressure remains constant for at most 0.5 s [seconds], in particular at most 0.2 s, preferably 0.1 s, particularly preferably never. In particular, the pressure is constant only when a switch is made between a fluid supply rate and a fluid discharge rate.

The statements concerning the first ventilation device, the second ventilation device, the control device, the first method and the second method can be transferred in each case to the other subjects of the present invention.

It is expressly noted that the control device may also be claimed independently of the ventilation device. The control device serves in particular to regulate the ventilation processes. It establishes which variables are used to control the ventilation process and which parameters (maximum/minimum pressure, maximum/minimum fluid supply rate and fluid discharge rate, etc.) are thereby monitored.

The invention and the technical field will be explained in more detail below on the basis of the figures. It should be noted that the figures show a particularly preferred embodiment variant of the invention, to which the invention is however not restricted. Here, identical components in the figures are denoted by the same reference signs. In the figures, in each case schematically:

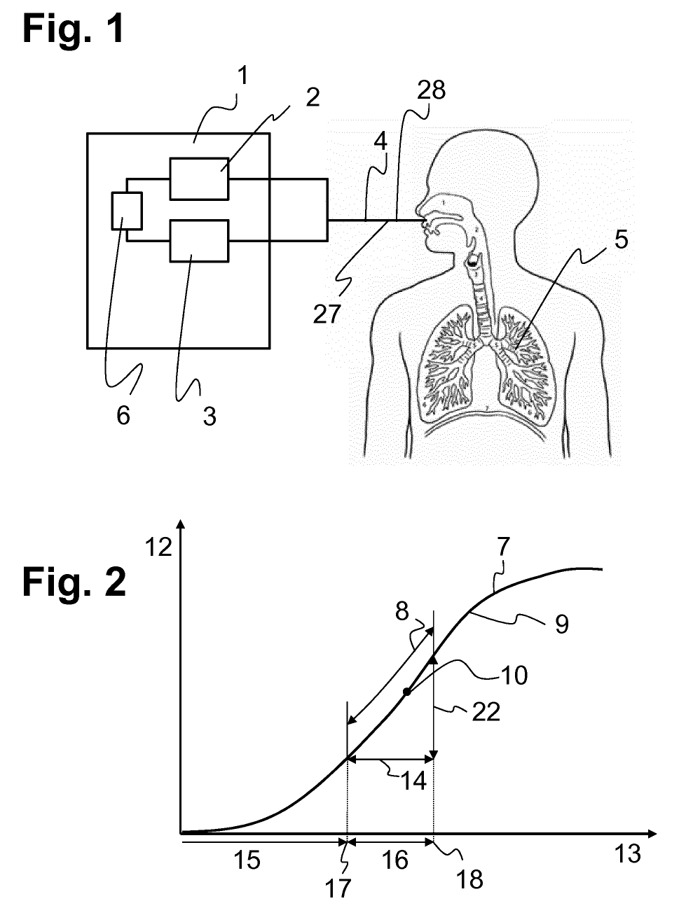

FIG. 1 shows a ventilation device and a patient;

FIG. 2 shows a profile of a compliance curve;

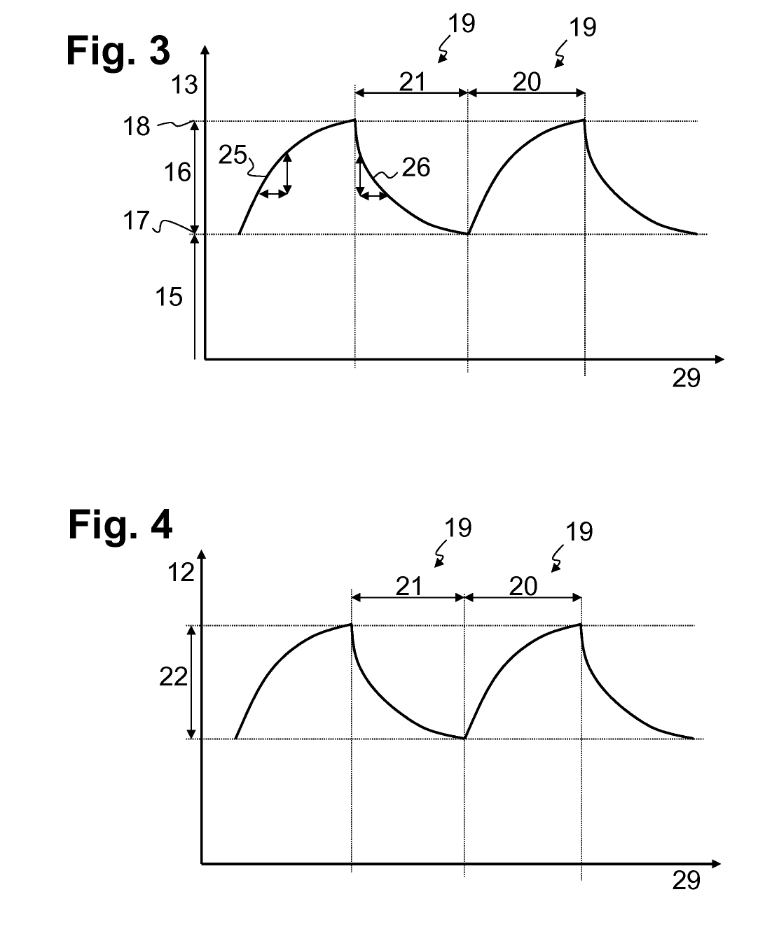

FIG. 3 shows ventilation processes in a pressure/time diagram;

FIG. 4 shows ventilation processes in a volume/time diagram;

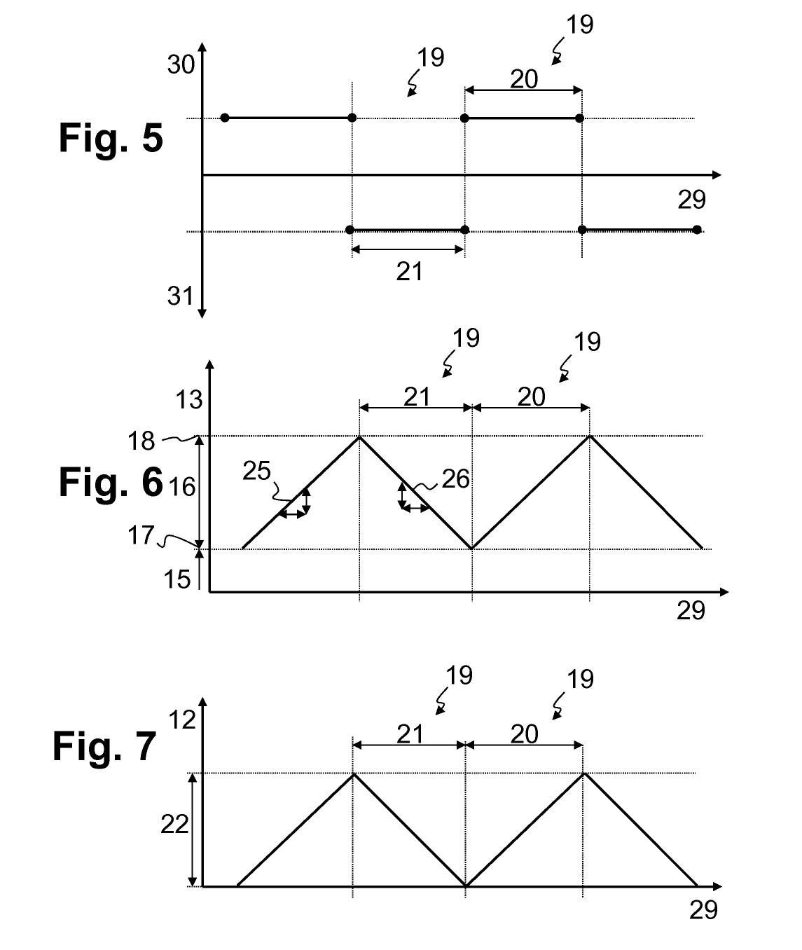

FIG. 5 shows ventilation processes in a fluid supply rate and fluid discharge rate/time diagram;

FIG. 6 shows ventilation processes in a further pressure/time diagram; and

FIG. 7 shows ventilation processes in a further volume/time diagram.

FIG. 1 shows a ventilation device 1 and a patient with at least one airway 5 or a lung. The ventilation device 1 comprises a fluid supply unit 2 and a fluid discharge unit 3 which are suitable for supplying a fluid 4 into an airway 5, that is to say into a lung part or into the lung, of a patient and for discharging the fluid 4 from this airway 5. The ventilation device 1 further comprises a control device 6 which, during a ventilation of the at least one airway 5 of the patient, that is to say the supply of a fluid 4 into the at least one airway 5 and/or the discharge of the fluid 4 from the at least one airway 5 by operation of the ventilation device 1, is suitable for determining or additionally for estimating a profile 7 of at least one subregion 8 of a compliance curve 9 of the at least one airway 5 by the supply and/or discharge of the fluid 4 from the at least one airway 5 and by determination of at least one value 10 of the compliance 11. Here, the ventilation device 1 is connected to the airway 5 of the patient via a catheter 27 with a lumen cross section 28 through which the fluid 4 can flow. The ventilation thus takes place, for example, via a single lumen, in particular using a gas flow reversal device.

FIG. 2 shows a profile 7 of a compliance curve 9 in a pressure/volume diagram. The pressure 13 is plotted on the horizontal axis, the volume 12 on the vertical axis. The profile 7 of the compliance curve 9 is to be determined individually for each patient. Moreover, the profile 7 may also change during a ventilation.

At least one value 10 of the compliance 11 is initially determined in the context of the method and by the ventilation device 1, wherein the following applies for the compliance 11: C=volume V 12/delta p 14 in milliliter/millibar. In the subregion 8 of the compliance curve 9 illustrated here, the absolute value of the compliance 11 is maximal. By determination or estimation of the profile 7 of the compliance curve 9, it is now possible to determine the position 15 of a pressure interval 16 with the pressures 17, 18 in which a tidal volume V_{T} 22 of the fluid 4 can be supplied to the at least one airway 5. These pressures 17, 18 are set on the ventilation device 1, such that at least one ventilation process 19, that is to say an inhalation 20 and/or an exhalation 21, takes place in each case with a tidal volume V_{T} 22 between these pressures P1 17 and P2 18.

FIG. 3 shows ventilation processes 19 in a pressure/time diagram. The time 29 is plotted on the horizontal axis, the pressure 13 on the vertical axis. The ventilation takes place in a pressure interval 16 between the pressures P1 17 and P2 18. The pressure rise 25, that is to say delta p/delta t, during the inhalation 20 is monitored and controlled. Moreover, the pressure drop 26, that is to say delta p/delta t, during the exhalation 21 is monitored and controlled.

FIG. 4 shows ventilation processes 19 in a volume/time diagram. The time 29 is plotted on the horizontal axis, the volume 12 on the vertical axis. The volume supplied to the airway 5 in the pressure interval 16 is designated as the tidal volume V_{T} 22. The profile of the curve in the volume/time diagram follows the profile of the pressure (see FIG. 3).

FIG. 5 shows ventilation processes 19 in a fluid supply rate and fluid discharge rate/time diagram. The time 29 is plotted on the horizontal axis, the fluid supply rate 30 (top, i.e. positive value) and fluid discharge rate 31 (bottom, i.e. negative value) on the vertical axis. The fluid supply rate 30 and the fluid discharge rate 31 are each constant and never zero. The fluid supply rate 30 and fluid discharge rate 31 can be set manually by the user on the ventilation device 1 or can be automatically regulated by the control logic unit of the ventilation device 1 or of the control device 6, wherein the pressure 13 is monitored. Alternatively, the pressure 13 (in the airway 5), as shown in FIG. 3, can be set e.g. by the user and monitored e.g. by the control logic unit, such that, at the ventilation frequency F 23 and/or the intended duration or time ratio of inhalation 20 and exhalation 21, the desired fluid supply rate 30 and fluid discharge rate 31 result from the set pressure 13.

In particular, the fluid supply rate 30 and fluid discharge rate 31 can be set via gas flow reversing elements already known from WO 2008/113752 A1 and WO 2015/004229 A1 as ventilation devices 1 which can be operated mechanically or manually.

FIG. 6 shows ventilation processes 19 in a further pressure/time diagram. The time 29 is plotted on the horizontal axis, the pressure 13 on the vertical axis. The ventilation takes place in a pressure interval 16 with the position 15 between the pressures P1 17 and P2 18. The pressure rise 25, that is to say delta p/delta t during the inhalation 20 is monitored and controlled. Moreover, the pressure drop 26, that is to say delta p/delta t, during the exhalation 21 is monitored and controlled. It will be seen here that the rise of the pressure profile is constant both during the inhalation 20 and also during the exhalation 21.

FIG. 7 shows ventilation processes 19 in a further volume/time diagram. The time 29 is plotted on the horizontal axis, the volume 12 on the vertical axis. The volume supplied to the airway 5 in the pressure interval 16 is designated as tidal volume V_{T} 22. The profile of the curve in the volume/time diagram follows the profile of the pressure (see FIG. 6). It will also be seen here that the rise of the volume profile is constant both during the inhalation 20 and also during the exhalation 21.

It will be seen from the ventilation processes 19 in FIGS. 3 to 7 that there are no ventilation pauses. The alternation between inhalation and exhalation takes place in each case without a pause.

### LIST OF REFERENCE SIGNS

1 ventilation device

2 fluid supply unit

3 fluid discharge unit

4 fluid

5 airway

6 control device

7 profile

8 subregion

9 compliance curve

10 value

11 compliance C

12 volume V

13 pressure

14 pressure difference delta p

15 position

16 pressure interval

17 pressure P1

18 pressure P2

19 ventilation process

20 inhalation

21 exhalation

22 tidal volume V_{T}

23 ventilation frequency F

24 pressure sensor

25 pressure rise

26 pressure drop

27 catheter

28 cross section

29 time +fluid supply rate −fluid discharge rate

1. The ventilation device for ventilating a patient, the ventilation device comprising a fluid supply unit or, additionally, a fluid discharge unit, which is suitable for supplying a fluid into at least one airway of a patient or for discharging the fluid from this airway; and a control device which, during a ventilation of the at least one airway, is suitable for determining or in addition for estimating a profile of at least one subregion of a compliance curve of the at least one airway by the supply and/or discharge of the fluid from the at least one airway and by determination of at least one value of the compliance; wherein the following applies for the compliance C:
C=V/delta p [milliliter/millibar];
wherein the compliance indicates how much fluid is introduced into the at least one airway or is removed from the airway, such that a pressure in the airway changes by a pressure difference delta p [millibar]; wherein the control device, taking into account the determined or additionally estimated profile of the at least one subregion of the compliance curve, determines a position of a pressure interval with the pressures P1 and P2 and sets the ventilation device such that at least one ventilation process takes place between these pressures P1 and P2 and an absolute value of the compliance of this ventilation process is as high as possible.

2. The ventilation device as claimed in claim 1, wherein the control device, at least during an inhalation or an exhalation of a ventilation process, determines a plurality of values for the compliance and from the plurality of values for compliance determines, for at least one subsequent ventilation process, the position of the pressure interval with the pressures P1 and P2, for which an absolute value of the compliance is as high as possible.

3. The ventilation device as claimed in claim 1, wherein the control device continuously determines values for the compliance, such that the position of the pressure interval with the pressures P1 and P2 can be newly determined optionally for each subsequent ventilation process or for a plurality of successive ventilation processes.

4. The ventilation device as claimed in claim 1, wherein the control device, as a function of the determined position of the pressure interval, of the pressure interval itself and of the determined compliance, determines at least for the subsequent ventilation process at least one of the following parameters:
a tidal volume V_{T} [milliliter],
a pressure P1 and a pressure P2 [millibar],
a ventilation frequency F [1/second].

5. The ventilation device as claimed in claim 1, wherein a pressure sensor is arranged inside the at least one airway, and the pressure is determined by a measurement inside the at least one airway.

6. The ventilation device as claimed in claim 1, wherein at least one pressure rise during an inhalation is controlled and limited by the ventilation device.

7. The ventilation device as claimed in claim 1, wherein at least one pressure drop, during an exhalation is controlled and limited by the ventilation device.

8. The ventilation device as claimed in claim 6, wherein the absolute value of the pressure rise is limited to at most 40 mbar/s [millibar/second].

9. A ventilation device for ventilating a patient, the ventilation device comprising a fluid supply unit or, additionally, a fluid discharge unit, which is suitable for supplying a fluid into at least one airway of a patient or for discharging the fluid from this airway; and a control device which, during a ventilation of the at least one airway, is suitable for regulating the ventilation; wherein the control device regulates at least one ventilation process such that a pressure in the at least one airway is continuously changed at least during an entire inhalation or at least during an entire exhalation, by continuous control of a fluid supply rate or of a fluid discharge rate during the ventilation process.

10. The ventilation device as claimed in claim 9, wherein the control device regulates at least one ventilation process such that there is a substantially constant fluid supply rate [milliliter/second] at least during an entire inhalation or there is a substantially constant fluid discharge rate [milliliter/second] at least during an entire exhalation.

11. The ventilation device as claimed in claim 9, wherein the control device regulates at least one ventilation process, which comprises at least one inhalation and one exhalation, such that a pressure in the at least one airway is changed continuously during the ventilation process.

12. The ventilation device as claimed in claim 11, wherein a continuous change of the pressure entails that the pressure remains constant for at most 0.5 s [seconds].

13. A method for operating a ventilation device, which is provided for ventilating a patient, said method comprising the following steps:
a) supplying a fluid into at least one airway of the patient and/or discharging the fluid from this airway by operation of the ventilation device;
b) determining or additionally estimating a profile of at least one subregion of a compliance curve of the at least one airway by the supply and/or discharge of the fluid in step a) and determination of at least one value of the compliance, wherein the following applies for the compliance:
C=V/delta p [milliliter/millibar];
wherein the compliance indicates how much fluid is introduced into the at least one airway or is removed from the airway, such that a pressure in the airway changes by a pressure difference delta p [millibar];
c) determining a position of a pressure interval with the pressures P1 and P2 along the profile of the at least one subregion of the compliance curve determined or additionally estimated in step b), wherein an absolute value of the compliance is as high as possible for a ventilation process performed in this pressure interval, i.e. an inhalation and/or an exhalation;
d) supplying and/or discharging the fluid within the pressure interval determined in step c), in at least one ventilation process following on from step c).

14. The method as claimed in claim 13, wherein in step b) a plurality of values for the compliance are determined at least during an inhalation or an exhalation of a ventilation process, such that in step c) it is possible to determine, for at least one subsequent ventilation process, the position of the pressure interval with the pressures P1 and P2 for which an absolute value of the compliance is as high as possible.

15. The method as claimed in claim 13, wherein the steps b), c) and d) are carried out continuously, such that the position of the pressure interval with the pressures P1 and P2 is newly determined optionally for each subsequent ventilation process or for a plurality of successive ventilation processes.

16. The method as claimed in claim 13, wherein, as a function of the position of the pressure interval determined in step c), of the pressure interval itself and of the determined compliance, at least one of the following parameters is determined at least for the subsequent ventilation process:
a tidal volume V_{T} [milliliter],
a pressure P1 (17) and a pressure P2 [millibar],
a ventilation frequency F [I/second].

17. The method as claimed in claim 13, wherein the pressure is determined by a measurement inside the at least one airway.

18. The method as claimed in claim 13, wherein at least one pressure rise, during an inhalation is controlled and limited.

19. The method as claimed in claim 13, wherein at least one pressure drop, during an exhalation is controlled and limited.

20. The method as claimed in claim 18, wherein the absolute value of the pressure rise is limited to at most 40 mbar/s [millibar/second].

21. The method as claimed in claim 13, wherein the patient is ventilated using a catheter which has a cross section of at most 30 mm² [square millimeters] for the passage of at least one fluid supplied during the inhalation.

22. A method for operating a ventilation device, which is provided for ventilating a patient, said method comprising at least the following steps:
a) supplying a fluid into at least one airway of the patient and/or discharging the fluid from this airway by operation of the ventilation device;
b) continuously changing a pressure in the at least one airway by continuously controlling a fluid supply rate and a fluid discharge rate during the ventilation processes.

23. The method as claimed in claim 22, wherein the continuous change of the pressure entails that the pressure remains constant for at most 0.5 s [seconds].

24. The method as claimed in claim 22, wherein there is a substantially constant fluid supply rate [milliliter/second] at least during an entire inhalation or there is a substantially constant fluid discharge rate [milliliter/second] at least during an entire exhalation.
